(19) United States
(12) Patent Application Publication
(10) Pub. No.: US 2016/0361507 A1
(43) Pub. Date: Dec. 15, 2016

(54) Nasal Delivery Device and Methods of Use

(71) Applicant: Bruce H. Levin, Philadelphia, PA (US)

(72) Inventor: Bruce H. Levin, Philadelphia, PA (US)

(21) Appl. No.: 15/181,079

(22) Filed: Jun. 13, 2016

Related U.S. Application Data
(60) Provisional application No. 62/174,165, filed on Jun. 11, 2015.

(51) Int. Cl. A61M 11/00; A61M 15/00; A61F 13/40; A61M 15/08

(52) U.S. Cl. CPC A61M 11/006; A61M 15/08; A61M 15/003; A61M 15/0003; A61M 35/006; A61M 15/009; A61M 15/0005; A61M 2206/12; A61M 2206/16; A61M 2206/14

(57) ABSTRACT

The present invention relates to a medical device for intranasal delivery of a medicament. The medicament may be any type of medicament suitable for nasal administration and delivery in the form of a spray. The present invention ensures that a complete dosage of the medicament is delivered, especially to specific areas in the nasal cavity, such as the rear of the nasal cavity where the SPG is located.

### RELATED APPLICATIONS

This application claims the benefit of U.S. Provisional Application No. 62/174,165 filed Jun. 11, 2016 and herein incorporated by reference.

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### BACKGROUND OF THE INVENTION

Nasal delivery of pharmaceutical products can be useful both for treating diseases or disorders in the nasal passages themselves and for treating systemic and/or neurological disorders. It has been observed, however, that particle or droplet size has significant impact on absorption when administering drugs via the nose and the nasal epithelia. Smaller droplets have been shown to promote better absorption into the body.

On the other hand, droplets that are too large fail to be absorbed and undesirably find their way into the pulmonary region.

Traditional devices for delivering drugs to the nasal cavity include syringed nose drops, pump spray devices, swabs, and propellant metered dose inhalers (MDI). These traditional devices have not generally been able to achieve the particle sizes necessary to maximize efficacy while helping mitigate undesired pulmonary absorption. For example, both eye dropper type devices and simple spray devices typically present medicament into the nasal cavity in a stream. The result is that much of the medicament simply runs out of the patient's nose or down the throat, and only a small amount of the drug is absorbed, with even less of the drug reaching the desired area in the nasal cavity.

Thus, there is a need for improved drug delivery systems to administer a desired dosage to the nasal epithelia or a predetermined area such as the sphenopalatine ganglion (hereinafter, the “SPG”).

### BRIEF SUMMARY OF THE INVENTION

In one embodiment, the present invention relates to a medical device for intranasal delivery of a medicament. The medicament may be any type of medicament suitable for nasal administration and delivery in the form of a spray.

In other embodiments, the present invention ensures that a complete dosage of the medicament is delivered, especially to specific areas in the nasal cavity such as the rear of the nasal cavity where the SPG is located.

A nasal drug delivery device according to the present invention includes a pump that supplies a fluid or powder containing a drug to a spray nozzle. The spray nozzle is adapted to create a spray plume hat has a particle size and distribution that increase the absorption of the drug at the desired site. In other embodiments, a powder is used instead of a fluid.

In another embodiment, the nozzle creates a vortex that may be used to coat the entire cavity with a tine mist of liquid or powder.

In yet other embodiments, the pressure supplied and the nozzle opening is adjustable to create a desired spray pattern or plume that may be concentrated towards a specific area.

In still another embodiment, the present invention provides a nozzle that imparts a circular or swirling motion to the medicament as it passes through the nozzle so as to accurately direct the medicament to a desired area. This may be accomplished by using one or more fins, spirals or grooves in the bore of the nozzle. In addition, one or more helical elongated members such as ribbon elements located in and extending around and along the bore may be used.

In yet a further embodiment, the present invention provides a reservoir to hold a liquid medicament, a pump connecting the reservoir to a spray port, and a swab having a passageway therein with an applicator at the distal end for applying a medicament to a desired area.

In further embodiments, the swab is anatomically contoured to target a specific location in the nasal cavity. A preferred targeted location is the SPG, which is relatively inaccessible as a result of being located below a region of epithelium in the posterior portion of the nasal cavity, inferior to and including the spheno-ethmoidal recess. As a result, the anatomical contouring may include three geometries. Up and back along the SPG, down and back along the SPG, and the middle of the SPG.

In yet other embodiments, the present invention is directed to delivery systems that administer single doses of one or more substances, for example a liquid, powder, or gel, to each nostril of a user. As used herein, the term “delivery system” is interchangeable with “delivery device” or “device.” The delivery systems of the present invention may deploy a pressurized container to hold and deliver a predetermined volume of substance to a particular destination with the administration, independent of the coordination of the user.

As used herein, the term “substance” includes but is not limited to one or more active-ingredient-containing substances wherein the active ingredient may be biologic agents such as a protein, peptide, vaccine, or an active pharmaceutical ingredient (“API”), for example a pharmaceutical drug such as a prescription drug, generic drug, or over-the-counter pharmaceutical, neutraceutical or homeopathic product. The substance may be in an aqueous, gel, powder, solution, emulsion, crystals or suspension form. As used herein, the term “substance” is interchangeable with the terms “drug,” “drug product,” “medication,” “liquid,” “biologic,” “active ingredient” or “API.” As used herein, an “active ingredient” or API is any component intended to furnish pharmacological activity or other direct effect in the diagnosis, cure, mitigation, treatment, or prevention of disease, or to affect the structure or any function of the body of humans or other animals. As used herein, the term “unit dosage form” is interchangeable with the terms “bottle,” “vial,” “unit-dose,” “dosage form,” “unit-dose vial,” “blister,” “dosage blister,” “ampule” or “container.”

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1 illustrates an embodiment of the present invention.

FIGS. 2A, 2B, 2C, 2D, 2E and-2F illustrate a spray pattern for an embodiment of the present invention.

FIG. 3A-3B illustrate structures for creating spray patterns that may be used with embodiment of the present invention.

FIG. 4 illustrates structures for creating spray patterns that may be used with embodiment of the present invention.

FIG. 5 illustrates another embodiment of the present invention.

FIG. 6 illustrates yet another embodiment of the present invention.

FIG. 7 illustrates a further embodiment of the present invention.

FIG. 8 illustrates yet another embodiment of the present invention.

FIGS. 9A, 9B, 9C, 9D, 9E, 9F, and 9G illustrate another embodiment of the present invention.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

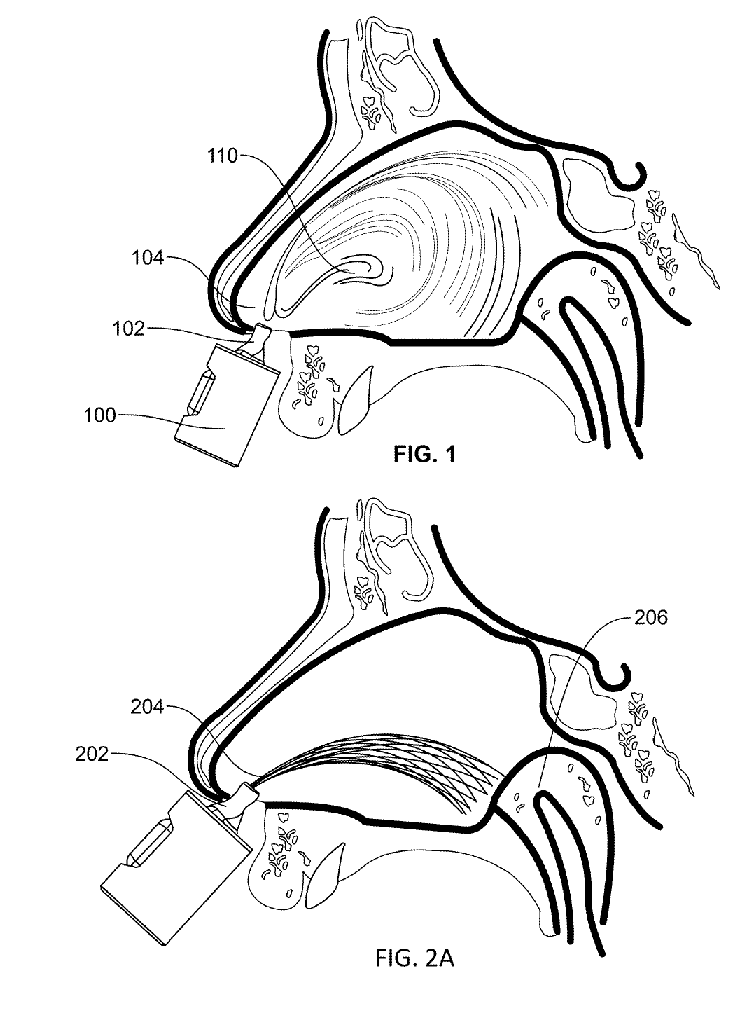

As shown in FIG. 1, in one embodiment, the present invention includes a housing 100 and nozzle 102. Nozzle 102 is configured to atomize the medicament into a fine mist. The nozzle can take any form known in the art. As shown, nozzle 102 is configured to bathe a substantial portion of nasal cavity 104 with a medicament 110. In other embodiments, a spray, which may be a vortex spray, may be used to cleanse and irrigate a desired area.

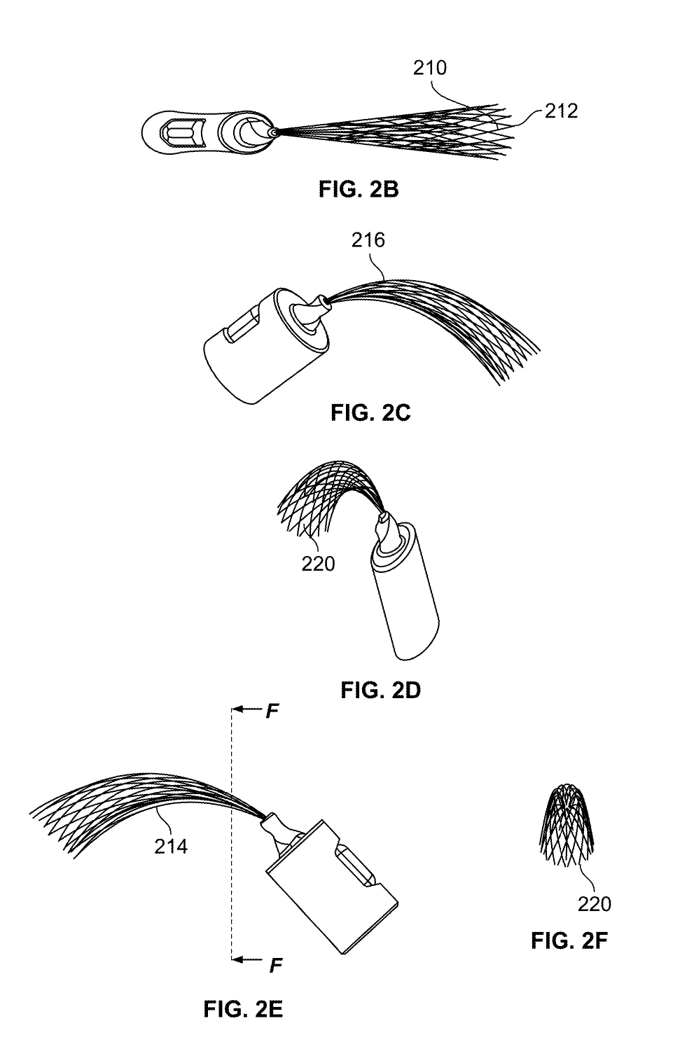

As shown in FIG. 2A, in an alternate embodiment, the spray output from nozzle 202 forms a plume 204 that advantageously is configured to cover a specific area within the nasal cavity such as SPG 206. As shown in FIGS. 2B-2F, plume 204 may have a predetermined width 210 at end 212, predetermined lower arc length 214, predetermined upper arc length 216, and predetermined cross-section 220. As shown, the plume may be preconfigured to target and cover a specific area in the nasal cavity.

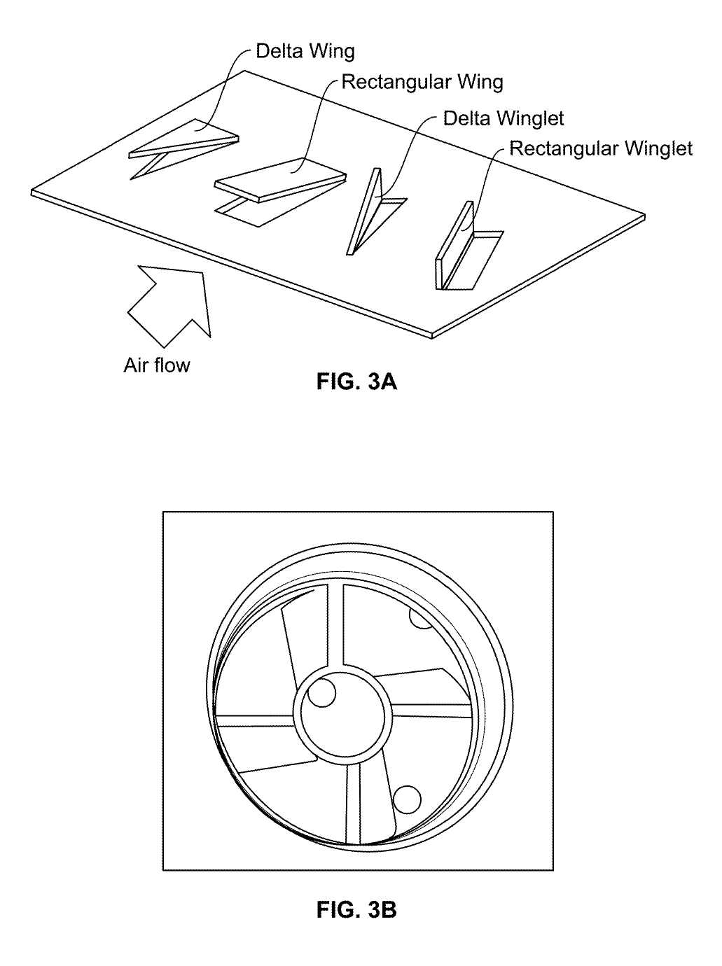

FIGS. 3A and 3B as well as FIG. 4, show various nozzle designs that may be used to generate the vortex or plume. As shown, one or more fins or wings may be located in the nozzle to create the circular plume. Other designs that may be used include extending along the length of the nozzle bore one or ore grooves that may take the form of rifling.

In use, the pressurized medicament is converted in the bore of the nozzle from a simple stream into a circular or swirling stream, which is positively projected toward the desired area for treatment.

Yet other embodiments include modifying the bore to include placing solid structures in the bore to alter the flow. Such shapes may include structures that form twisting, conical, spiral, nautilus, rain droplet shaped, hi-conical, disc-like, biconvex, biconvex patterns, spherical, and partially spherical with a trailing edge as well as other patterns.

In other embodiments, the present invention provides catheters, medical structures, medical surfaces, and cannulas that are stealth like, or curve stealth like and any combination therein. Inner or outer surfaces may have depressions or relief shapes to alter fluid flow over a surface, to facilitate laminar, vortex or other flow. The surfaces may be applied to valve or valve like structures, conduits, catheters, stents, blood vessels, lumens, artificial organ structures, drains, bypass, perfusion or other pumps, artificial heart or chambers, vessels, and attachments thereof.

Yet other embodiments include modifying the surface of the object to alter fluid flow over the surface of a medical object to be inserted into a fluid stream to facilitate laminar, vortex or other flow. To modify the fluid flow around the object, preconfigured shapes are placed around the object. Preferred shapes include, but are not limited to, placing solid structures such as tubes as well as other structures that form twisting, conical, spiral, nautilus, rain-droplet-shaped, hi-conical, disc-like, biconvex, and biconvex patterns, tube, partially spherical, partially spherical with trailing edge, and spermatozoic morphology patterns.

Altered shape or surface morphologies will alter fluid flow characteristics and velocity, surface friction and durability, wear, surface trauma, vessel trauma, implant stability, catheter oscillations, sensor artifact, eddy currents, wave reflection, red cell and other cell trauma/integrity, clotting characteristics, platelet adhesion activation, inflammatory mediator activation, and thrombogenicity and altered infectious risk.

The angle and mode of vessel insertion will alter flow characteristics. The interface between catheter and hub also will play a role in peri-catheter flow dynamics.

As shown in FIG. 5, another embodiment of the present invention provides a device 500 that has a retractable nozzle. Device 500 has a housing 502 that contains a medicament, and, when squeezed, the medicament advances through tube 504 and causes nozzle 506 to extend distally toward a desired area such as the SPG. Nozzle 506 is preferably made of an elastic material. This increases patient comfort by reducing the overall length of device 500 during insertion into the nasal cavity.

Nozzle 506 may use the vortex spray nozzles described above. In addition, the retractable nozzle may extend straight out, off to a side, extend in a curved path or may expand out into a bulbous shape or other shapes which have an expanded surface area.

In other embodiments the pressure provides a force that causes the nozzle to articulate, oscillate or move in a predetermined pattern to bathe the entire nasal cavity. The nozzle may also be adapted to move in a predetermined pattern that results in applying medicament or a solution to a particular area in the nasal cavity.

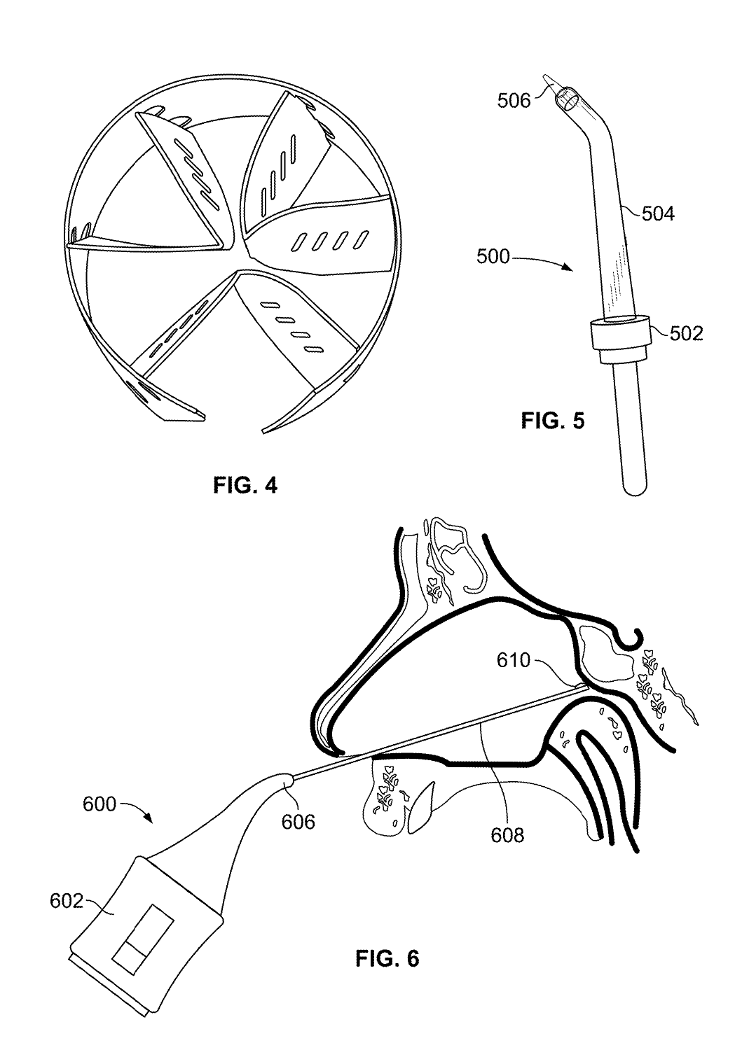

As shown in FIG. 6, in yet another embodiment, the present invention provides an anatomically friendly swab that may be used to target a specific area in the nasal cavity such as the SPG. Device 600 includes a housing 602 for containing medicament, which may be pressurized. Nozzle 606 may be connected to a tube 608, which includes an applicator 610 or tip that is semi-spherical in shape. Applicator 610 is designed to create a controlled delivery. This may be accomplished by designing the applicator to become saturated, sponge-like, for a direct application to the desired area. The applicator may also be designed to be expandable upon saturation so as to present a minimally invasive area for insertion and then an increased surface area for applying the medicament to the desired area. Also, the applicator may include openings that may be used to create a gentle, controlled spray or flow such as seeping or oozing. Thus, as medicament is delivered to applicator 610, it may be sprayed on or delivered to a desired area in a controlled manner, directly applied, or both. Alternately, the tip may be saturated with medicament and then applied to a specific area in the nasal cavity such as the SPG in a controlled manner. In other embodiments, the tube may be trimmed to length prior to attachment to the nozzle. In addition, the tube may be bendable to permit it to be configured into a desired shape to aid in the placement of the applicator inside the nasal cavity.

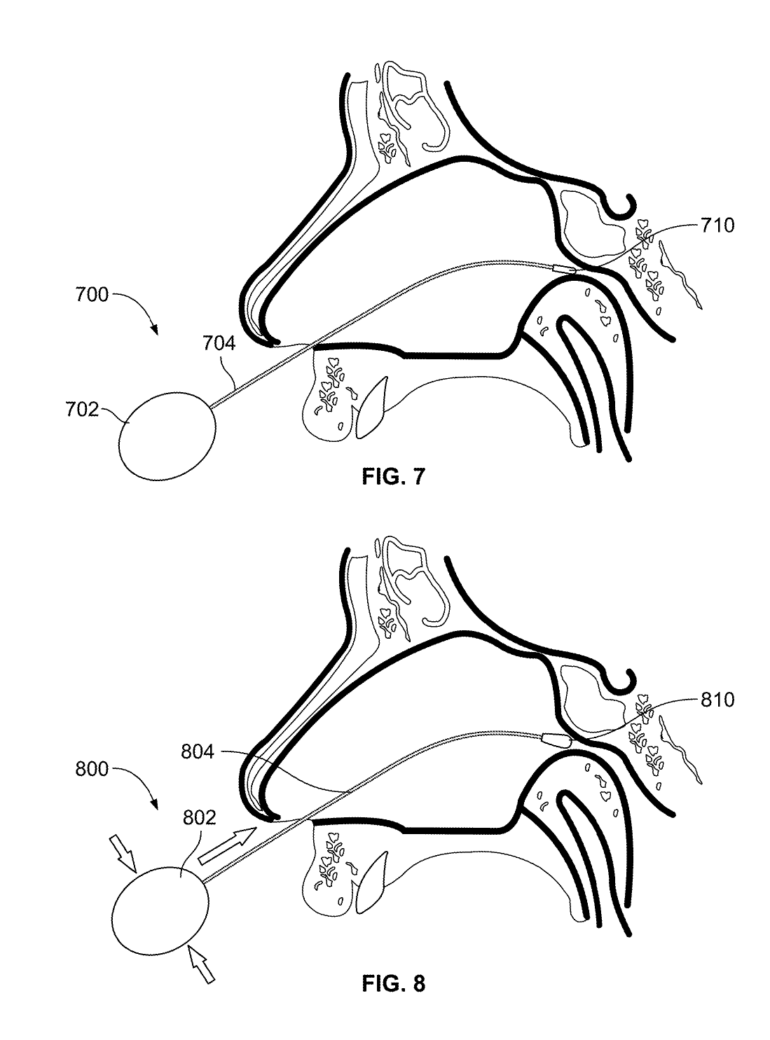

As shown in FIG. 7, in yet another embodiment, the present invention provides an anatomically friendly swab that may be used to target a specific area in the nasal cavity, such as the SPG. Device 700 includes a pump 702 for containing medicament, and expelling the medicament under pressure. Tube 704 is connected to pump 702 and further includes an applicator 710 or tip that is conical in shape. Applicator 710 is designed to create a controlled delivery. This may be accomplished by designing the applicator to become saturated, sponge-like, for a direct application to the desired area. The applicator may also be designed to be expandable upon saturation so as to present a minimally invasive area for insertion and then an increased surface area for applying the medicament to the desired area. Also, the applicator may include openings that may be used to create a gentle, controlled spray or flow such as seeping or oozing. Thus, as medicament is delivered to applicator 710, it may be sprayed on or delivered to a desired area in a controlled manner, directly applied, or both. Alternately, the tip may be saturated with medicament and then applied to a specific area in the nasal cavity such as the SPG in a controlled manner. In other embodiments, the tube may be trimmed to length prior to attachment to the nozzle. In addition, the tube may be bendable to permit it to be configured into a desired shape to aid in the placement of the applicator inside the nasal cavity.

As shown in FIG. 8, in yet another embodiment, the present invention provides an anatomically friendly swab that may be used to target a specific area in the nasal cavity, such as the SPG. Device 800 includes a pump 802 for containing medicament, and expelling the medicament under pressure. Tube 804 is connected to pump 802 and further includes an applicator 810 that is oval-like in shape. Applicator 810 is designed to create a controlled delivery. This may be accomplished by designing the applicator to become saturated, sponge-like, for a direct application to the desired area. The applicator may also be designed to be expandable upon saturation so as to present a minimally invasive area for insertion and then an increased surface area for applying the medicament to the desired area. Also, the applicator may include openings that may be used to create a gentle, controlled spray or flow such as seeping or oozing. Thus, as medicament is delivered to applicator 810, it may be sprayed on or delivered to a desired area in a controlled manner, directly applied, or both. Alternately, the tip may be saturated with medicament and then applied to a specific area in the nasal cavity such as the SPG in a controlled manner. In other embodiments, the tube may be trimmed to length prior to attachment to the nozzle. In addition, the tube may be bendable to permit it to be configured into a desired shape to aid in the placement of the applicator inside the nasal cavity.

In other embodiments, the nozzles of the various disclosed embodiments may be configured to create a vortex of fluid, powder, or combination thereof. Creating a vortex permits a fluid, air or spray to entraining medicaments or other desired substances. The vortex also creates a precise flow that may be targeted with more precision to a desired location. As shown in the figures, one possible way to create a vortex is to include fins inside a nozzle.

In other embodiments, the present invention provides a novel medicament mixing system. The system includes preloaded dosages of individual medicaments that may be combined together to produce a desired compound. In one preferred embodiment, the dosages are held in individual containers or cartridges that are placed in a mixing chamber, opened, and then mixed. The mixture may then be dispensed.

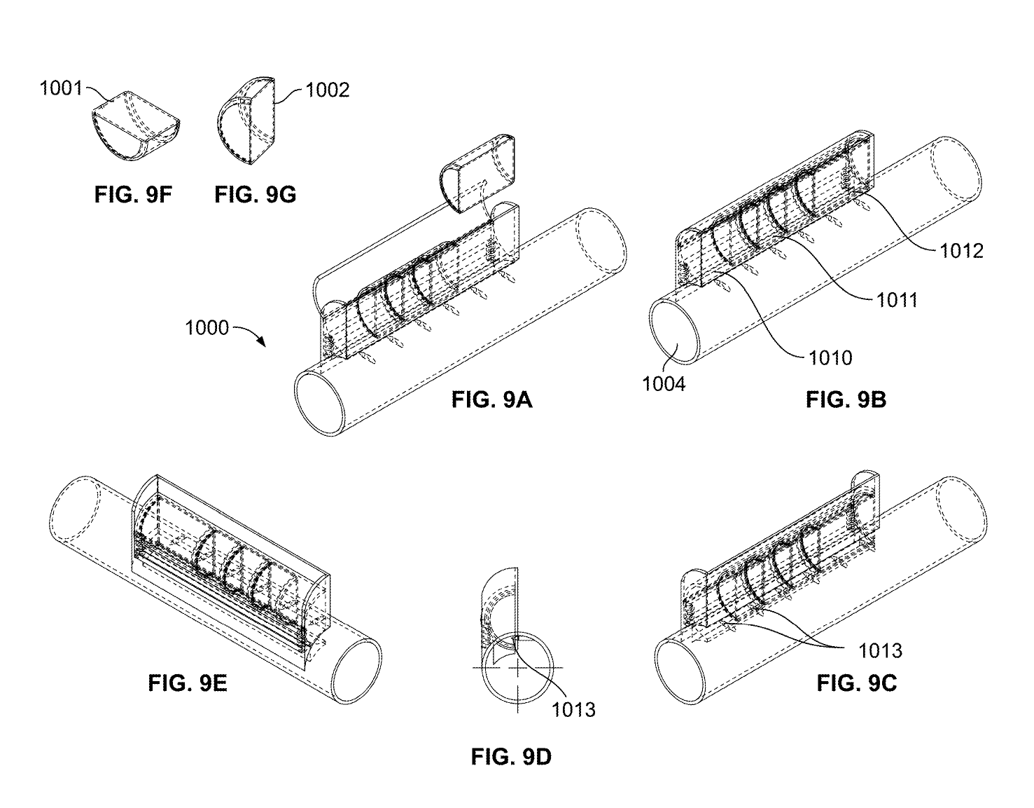

FIGS. 9A-9G depict a preferred embodiment. As shown, system 1000 includes one or more cartridges 1001-1002 that may be hollow semi-cylinder and filled with medicament so as to fit inside mixing chamber 1004. The medicaments in containers 1001-1002 may be prefilled with predetermined dosages that are combined together as described in further detail below to arrive at a desired mixture. In addition, other complimentary shapes for the cartridges and mixing chamber may be used. The cartridges may be sealed with foil or a plastic membrane that may be punctured or opened when desired to release the contents of the cartridge into the mixing chamber.

System 1000 may be used as follows: Step 1: cartridges of multiple and/or varying capacities (widths) are dropped into the loading chamber of the mixing chamber; Step 2: the loading chamber cover is closed; Step 3: spring loaded loading chamber (with cartridges) is pressed down causing one or more knife edges 1010-1013 at the mixing chamber opening to cut the cartridges sealing the membranes and allowing medicament to fall into mixing tube or chamber 1004.

The spring loaded chamber is released, in turn, sealing the mixing chamber. System 1000 now resembles the same state as Step 2 but with medicament in the mixing chamber. System 1000 may then be agitated to achieve a desired mixture.

The mixing chamber can be incorporated into a syringe, oral dosing, breathing, nasal or other delivery system as determined by an administrator and/or medicament manufacturer. Accordingly, alternate embodiments including cartridges that may be placed in a rotating, spinning or moveable housing such as a cylinder. The cylindrical housing is rotated to load a selected cartridge, and then further rotated to bring the cartridge in a position to allow the contents to be dispensed into a mixing chamber. Once the desired mixture is obtained, the device is configured to allow the medicament to be dispensed.

Other embodiments concern providing a device that is adapted to load the cartridges serially or in a linear fashion at one end, activated and mixed by a plunger, and then deliver from an opposing outlet. In yet other embodiments, the chamber is configured to function like a Y-connector that receives a plurality of cartridges, which may be individually dispensed into a common mixing chamber prior to administering the medicament.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above described embodiments, methods, and examples, but by all embodiments and methods within the scope and spirit of the disclosure.

What is claimed is:

1. A drug delivery system comprising: a housing configured to contain a medicament; a drug delivery mechanism to expel medicament from a nozzle; and said nozzle having a bore and configured to direct the medicament to a predetermined area in the nasal passageway.

2. The drug delivery system of claim 1 wherein said nozzle creates a vortex of spray or a preconfigured plume.

3. The drug delivery system of claim 2 wherein said plume is circular.

4. The drug delivery system of claim 1 wherein said drug delivery mechanism is configured to have an adjustable pressure and said nozzle has an adjustable opening.

5. The drug delivery system of claim 1 wherein said drug delivery mechanism is a pump.

6. The drug delivery system of claim 1 wherein said nozzle contains one or more fins, spirals, helical elongated members, rifling or grooves in the bore of the nozzle.

7. The drug delivery system of claim 1 wherein said nozzle contains solid structures in the bore to alter the flow.

8. The drug delivery system of claim 7 wherein said solid structures are conical, spiral, nautilus, rain droplet shaped, bi-conical, disc-like, biconvex, spherical, or partially spherical with a trailing edge.

9. The drug delivery system of claim 1 wherein said drug delivery mechanism is a manual pump.

10. The drug delivery system of claim 1 wherein said nozzle is separated from said drug delivery mechanism in a spaced apart relationship by a tube.

11. The drug delivery system of claim 10 wherein said tube and nozzle are contoured to target a specific location in the nasal cavity

12. The drug delivery system of claim 1 wherein said nozzle is configured to atomize the medicament.

13. The drug delivery system of claim 2 wherein said vortex of spray or said preconfigured plume has a predetermined width at the end, a predetermined lower arc length, a predetermined upper arc length, and a predetermined cross-section.

14. The drug delivery system of claim 1 wherein said nozzle is in a retracted position when inserted and extends outwardly for delivery of the medicament.

15. The drug delivery system of claim 1 wherein said nozzle expands into a shape having an increased surface area for delivery of the medicament.

16. The drug delivery system of claim 15 wherein said nozzle is bulbous, semi-spherical, conical or oval in shape.

17. The drug delivery system of claim 1 wherein said medicament is contained in individual containers.

18. The drug delivery system of claim 17 further including a mixing chamber adapted to receive one or more of said containers wherein said medicament is mixed prior to administration.

19. The drug delivery system of claim 18 wherein said containers and said mixing chamber are complimentary in shape.

20. The drug delivery system of claim 17 wherein said containers have a membrane that may be punctured or opened when desired to release the contents of the container into the mixing chamber.

21. The drug delivery system of claim 10 wherein said tube and nozzle are contoured to target a specific location in the nasal cavity by moving up and back along the SPG, down and back along the SPG, or the middle of the SPG.
